(19) United States
(12) Patent Application Publication
(10) Pub. No.: US 2018/0318338 A1
(43) Pub. Date: Nov. 8, 2018

(54) METHODS AND COMPOSITIONS FOR THE PREVENTION OF SUICIDE, HOMICIDE AND SELF-HARMING BEHAVIORS

(71) Applicant: THE MCLEAN HOSPITAL CORPORATION, BELMONT, MA (US)

(72) Inventor: Edward G. MELONI, Needham, MA (US)

(73) Assignee: THE MCLEAN HOSPITAL CORPORATION, BELMONT, MA (US)

(21) Appl. No.: 15/774,770

(22) PCT Filed: Nov. 9, 2016
(86) PCT No.: PCT/US16/61117

§ 371 Date: May 9, 2018

Related U.S. Application Data
(60) Provisional application No. 62/252,703, filed on Nov. 9, 2015.

(51) Int. Cl. A61K 33/00; A61K 9/00; A61M 16/12; A61M 16/20; A61M 16/06

(52) U.S. Cl. CPC A61K 33/00; A61K 9/0073; A61M 16/12; A61M 2016/003; A61M 16/06; A61M 2205/3592; A61M 16/20

(57) ABSTRACT

Provided herein are methods and compositions for the treatment and/or prevention of: suicide, homicide, and/or self-harming behavior, comprising administration of a composition comprising xenon.

### FIELD OF THE DISCLOSURE

The present disclosure relates to the prevention of suicide, homicide and self-harming behaviors.

### BACKGROUND

Xenon gas is currently used in humans as an anesthetic with a very safe profile. In addition, xenon has been shown to exert a neuroprotective effect, for example, in brain injury. Xenon has a fast “on-off” effect as it rapidly enters the brain during administration and is quickly cleared after discontinuing administration. Xenon acts through a number of mechanisms in the brain, including functioning as an antagonist of the glycine site on the NMDA receptor effectively reducing excitatory postsynaptic currents, calcium influx, and associated changes in synaptic plasticity. Xenon also affects non-NMDA glutamatergic:a-amino-3 hydroxy-5-methyl-4-isoxazolepropionic acid (AMPA) receptors, two-pore domain potassium channels (TREK-1), ATP-sensitive potassium channels (KATP), several nicotinic cholinergic receptors (alpha4beta2-alpha7- and alpha4beta4 subunit containing receptors), serotonin type 3 receptors (5-HT3), strychnine-insensitive inhibitory glycine receptors (GlyRs), and tissue plasminogen activator (tPA) and plasmin. Xenon also increases pro-survival factors such as BDNF and bcl2, and may inhibit pro-inflammatory cytokines such as tumor necrosis factor a and interleukin 1β.

### SUMMARY

Provided herein are methods and compositions for the treatment and/or prevention of: suicide, homicide, and/or self-harming behavior, comprising administration of a composition comprising xenon gas.

Accordingly, one aspect provided herein relates to a method for reducing the intensity of thoughts and/or feelings associated with suicide, self-harm, or homicide, the method comprising: administering a xenon composition to a subject in need thereof, thereby reducing the intensity of the thoughts and/or feelings.

In one embodiment of this aspect and all other aspects provided herein, the xenon composition is self-administered.

In another embodiment of this aspect and all other aspects provided herein, the xenon composition is administered by a second individual.

In another embodiment of this aspect and all other aspects provided herein, the second individual comprises an emergency responder, a relative, a spouse, a friend, a co-worker, or a health care professional.

In another embodiment of this aspect and all other aspects provided herein, the xenon composition comprises at least 0.5-35% xenon by volume.

In another embodiment of this aspect and all other aspects provided herein, the xenon composition further comprises air, nitrogen, oxygen, or a combination thereof.

In another embodiment of this aspect and all other aspects provided herein, the administration of xenon is a single exposure to xenon.

In another embodiment of this aspect and all other aspects provided herein, the administration of xenon is repeated as necessary until the intensity of the thoughts and/or feelings is reduced by at least 20%.

In another embodiment of this aspect and all other aspects provided herein, the administration of xenon is repeated as necessary until the intensity of the thoughts and/or feelings are reduced to a tolerable level.

In another embodiment of this aspect and all other aspects provided herein, the tolerable level is a level where the subject feels able to control or prevent self-harming or homicidal actions.

In another embodiment of this aspect and all other aspects provided herein, the xenon composition is administered by inhalation.

In another embodiment of this aspect and all other aspects provided herein, the xenon composition is not administered during a psychotherapy session.

Another aspect provided herein relates to a device for self-administration of xenon, the device comprising: (a) a canister containing a xenon gas stored at a high-pressure level; (b) a regulator in which the xenon gas is transformed from the high-pressure level to a breathable level; (c) a face mask communicatively coupled to the canister and the regulator and through which the xenon composition is externally administered, the face mask being connected to the canister and the regulator such that the xenon gas flows from the canister, through the regulator, and into the face mask; and (d) a medication event monitoring system communicatively coupled with the canister and configured to detect and/or determine changes in the xenon gas.

In one embodiment of this aspect and all other aspects provided herein, the device further comprises a push button configured to activate xenon gas flow from the canister to the face mask.

In another embodiment of this aspect and all other aspects provided herein, the device further comprises a graded flowmeter readout indicative of xenon gas conditions including at least one of: xenon gas flow and/or the remaining level of xenon gas.

In another embodiment of this aspect and all other aspects provided herein, the medication event monitoring system is configured to activate with each device use.

In another embodiment of this aspect and all other aspects provided herein, the medication event monitoring system further includes a wireless connection.

In another embodiment of this aspect and all other aspects provided herein, the wireless connection includes at least one of a Bluetooth™ connection and a Wi-Fi connection.

In another embodiment of this aspect and all other aspects provided herein, the medication event monitoring system is configured to send a status report to a desired individual (e.g., spouse, parent, therapist, etc.) or emergency personnel (e.g., 911 personnel, emergency medical services (EMS), ambulance, fire, etc.).

Another aspect provided herein relates to a self-contained breathing apparatus for on-demand inhalation of a therapeutic xenon gas, the apparatus comprising: (i) a replaceable canister including a mixture of high-pressure gas that contains air and a therapeutic xenon gas, the xenon gas being in the range of about 5-35% of the mixture; (ii)a built-in regulator connected to the canister and configured to receive and transform the mixture from the high-pressure gas to a breathable level gas; (iii) a face mask connected to the built-in regulator, the face mask receiving the breathable level gas of the mixture; (iv) a push-button mounted to the canister and configured to activate, upon pressing, flow of the mixture from the canister to the face mask; (v) a graded flowmeter readout connected to the canister and indicative of changes in the mixture, the changes including flow of the mixture and a remaining amount of the mixture in the canister; and (vi) a medication event monitoring system (MEMS) mounted at least in part on the canister, the MEMS being communicatively coupled to the canister and to a remote computer, the MEMS being activated with each pressing of the push-button, the MEMS reporting one or more use aspects of the mixture.

Another aspect provided herein relates to a method for treating or preventing an act of suicide, homicide or self-harming behavior, the method comprising: administering a xenon composition to a subject, thereby treating or preventing the act of suicide, homicide, or self-harming behavior.

In one embodiment of this aspect and all other aspects provided herein, the subject is determined to be at high risk for suicide, homicide or self-harming behavior.

In another embodiment of this aspect and all other aspects provided herein, the subject is determined to be at high risk for suicide, homicide or self-harming behavior based on self-reporting by the subject or as diagnosed by a healthcare professional using a clinical test.

In another embodiment of this aspect and all other aspects provided herein, the subject is diagnosed as having a high risk for suicide when the subject has a Columbia Suicide Severity Risk Scale (C-SSRS) level of at least Category 5.

In another embodiment of this aspect and all other aspects provided herein, the subject is diagnosed as having a high risk for homicide when the subject is determined to have a Key to Danger level of at least 4 on the Centers for Disease Control and Prevention's Danger Assessment Test.

In another embodiment of this aspect and all other aspects provided herein, the subject is diagnosed has having a high risk for self-harming behavior based on a past self-harming event or a score of at least 1 on the Self-Harm Inventory test.

In another embodiment of this aspect and all other aspects provided herein, the subject is in emotional and/or physical distress.

In another embodiment of this aspect and all other aspects provided herein, the subject is interrupted in an attempt of suicide, homicide or self-harming behavior.

In another embodiment of this aspect and all other aspects provided herein, the subject is holding a means for suicide, homicide, or self-harming behavior.

In another embodiment of this aspect and all other aspects provided herein, the means for suicide, homicide, or self-harming behavior comprises a firearm, a cutting utensil, a poison, a vial of drugs, an accelerant, an ignition source, a flammable liquid or gas, an asphyxiant, access to a ledge, bridge, pillar, or outcropping for jumping from a height, a ligature, a noose, material for electrocution, explosive material, access to a road or freeway for vehicular suicide, access to train tracks, suicide by cop, or access to water for drowning.

In another embodiment of this aspect and all other aspects provided herein, the subject is further administered an antidepressant, anti-anxiety, or anti-psychotic medication.

In another embodiment of this aspect and all other aspects provided herein, the antidepressant, anti-anxiety, or anti-psychotic medication is formulated for aerosol delivery.

In another embodiment of this aspect and all other aspects provided herein, the xenon composition is self-administered or administered by another individual.

In another embodiment of this aspect and all other aspects provided herein, the xenon composition comprises 0.5 to 35% xenon.

Another aspect described herein relates to a method of decreasing suicidal ideation in a subject having enhanced levels of at least one metabolite generated by indolamine 2, 3 deoxygenase comprising administration of 0.5-35% xenon gas.

In one embodiment of this aspect and all other aspects provided herein, the at least one metabolite is quinolinic acid or kynurenine.

In another embodiment of this aspect and all other aspects provided herein, the enhanced levels are higher than age-matched controls.

In another embodiment of this aspect and all other aspects provided herein, the xenon composition is administered by inhalation.

Another aspect provided herein relates to a method of decreasing suicidal ideation in a subject comprising: (i) measuring levels of at least one metabolite generated by indolamine 2, 3, deoxygenase in a biological sample obtained from a subject; and (ii) administering 0.5-35% xenon gas to the subject, thereby decreasing suicidal ideation.

In one embodiment of this aspect and all other aspects provided herein, the at least one metabolite is quinolinic acid or kynurenine.

In another embodiment of this aspect and all other aspects provided herein, the enhanced levels are higher than age-matched controls.

In another embodiment of this aspect and all other aspects provided herein, the xenon composition is administered by inhalation.

### BRIEF DESCRIPTION OF THE FIGURES

FIG. 1 An exemplary xenon self-administration device for on-demand inhalation of therapeutic xenon gas.

### DETAILED DESCRIPTION

### Definitions

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein and includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. In another embodiment, the subject is a domesticated animal including companion animals (e.g., dogs, cats, rats, guinea pigs, hamsters etc.).

The term “chronic administration” means the administration of an agent (e.g., a xenon composition as described herein) on a periodic basis (e.g., at least once a day, twice a day, three times a day, four times a day, at least once a week, twice a week, three times a week, four times a week, five times a week, six times a week, seven times a week, once a month, twice a month, three times a month, and four times a month) over an extended period of time (e.g., at least one month, two months, three months, four months, five months, six months, seven months, eight months, nine months, ten months, eleven months, one year, two years, three years, four years, five years, six years, seven years, eight years, nine years, or ten years). The periodic administration of the agent (e.g., a xenon composition as described herein) can be performed over a continuous period of time (as described herein) or can be administered as a bolus (e.g., inhalation of a single dose of xenon gas or administration of a dosage of a xenon composition (e.g., a nanoparticle or nanosponge).

The phrase “continuous period of time” means at least 5 minutes, (e.g., at least 10 minutes, at least 15 minutes, 30 minutes, 45 minutes, 1 hour, 2 hours, 3 hours, 4 hours, 5 hours, 6 hours, 7 hours, 8 hours, 9 hours, 10 hours, 11 hours, or 12 hours, overnight, or between 5 and 13 hours).

As used herein, “acute” administration of a xenon composition means a single exposure within an extended time period of the subject to the therapeutically effective amount of xenon. In conjunction with this definition of “acute”, an extended time period is defined as four days or longer, e.g., once-weekly administration of a xenon composition constitutes acute administration. Administering a dose of a xenon composition to a subject, followed by a second dose 24 hours later, does not constitute acute dosing. Repeated administration or self-administration to achieve a desired effect by administering at least a second exposure can still be considered “acute” administration when repeated dosing is required to titrate a dose to reduce the intensity of an episode of suicidality, homicidality, or self-harming behavior. In some embodiments, administration of a xenon composition occurs when the subject is in emotional and/or physical distress, is displaying the means for suicide, homicide, or self-harming behavior, or is verbally threatening suicide, homicide, or self-harming behavior. In such embodiments, the decision to administer a xenon composition can be made by a relative, friend, co-worker, spouse, healthcare professional, emergency responder or a good Samaritan and need not be consensual on the part of the subject.

As used herein, “a subject at risk for suicide, homicide, or self-harm” can refer to a subject diagnosed by one skilled in the art such as, for example, a clinician, using established protocols and methods for diagnosing suicidality, homicidality, or self-harming behaviors. Such methods can include, for example, rigorous clinical interview using clinical standards for assessing and diagnosing whether a subject is at risk for suicide. Suicidality diagnosis can be established using, for example, questionnaires to identify suicidal ideation. Diagnosis can include diagnostic assessment using psychiatric rating scales including, for example, the Hamilton Rating Scale for Depression (HAMD-17), which includes a suicidal ideation rating item, Beck Scale for suicide ideation, Columbia Suicide Severity Rating Scale, The Kessler Psychological Distress Scale, and combinations thereof. In some embodiments, a subject can determine their own risk for suicide by assessing or noting the intensity of feelings of suicide, homicide or self-harm, and can self-administer a xenon composition accordingly. In some embodiments, a healthcare professional or clinician can determine whether a subject is at risk for suicide, homicide, or self-harm and provide or prescribe a xenon composition to the subject if they are determined to be high risk or very high risk of suicide, homicide, or self-harm based on an accepted clinical test or as described herein. In one embodiment, the subject at risk for suicide, homicide, or self-harm comprises enhanced levels of at least one metabolite of indolamine 2, 3, deoxygenase (e.g., quinolinic acid, kynurenine) or increased levels of platelet 5HT2A receptor, or CSF SHIAA.

As used herein, the phrase “intensity of thoughts and/or feelings associated with suicide, homicide or self-harm” refers to the self-reported or clinically assessed degree of thoughts and/or feelings associated with suicide, homicide or self-harm. The intensity of thoughts and/or feelings is subjective and will vary from person to person, as well as during different periods of e.g., life, stress, etc. However, the ability to tolerate such thoughts or feelings without acting upon them will also vary from person to person, such that self-reporting of e.g., how tolerable the thoughts and/or feelings are gains significance relative to simply reporting the thoughts and/or feelings. The intensity can be determined by self-reporting by the subject or by one of skill in the art using a clinically relevant questionnaire or scale. However, it can also be estimated on a scale of 1 to 10, where 1 is little to no thoughts/feelings of such suicide, homicide, or self-harm, while 10 would reflect a feeling of powerlessness and inability to control an act of suicide, homicide or self-harm. Similarly, the thoughts/feelings can be estimated using a percentage from 1% to 100%. Each of these methods will rely on accurate self-reporting by the subject themselves. Alternatively, the subject can express the intensity of their thoughts/feelings as “tolerable” vs. “intolerable.”

As used herein, the terms “tolerable” or “tolerable level” refer to thoughts and/or feelings of suicide, self-harm or homicide at an intensity in which the subject may find uncomfortable, but are still tolerable in that the subject does not feel the need to act on such thoughts/feelings. Conversely, the terms “intolerable” and “intolerable level” refer to thoughts/feelings at an intensity that the subject feels an urge, a strong desire, or a need to act on such thoughts/feelings or that the subject feels powerless to control such an urge, desire, or need.

As used herein, the term “at least one metabolite generated by indolamine 2,3 deoxygenase” refers to the generation of e.g., quinolinic acid or kynurenine through the tryptophan catabolism pathway. Indolamine 2, 3, deoxygenase (gene name: IDO1, Gene Card I.D. No: GC08P039891) catalyzes the degradation of the essential amino acid L-tryptophan to N-kynurenine, the first and rate-limiting step of tryptophan catabolism through the kynurenine pathway.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase” or “enhance” or “activate” are all used herein to generally mean an increase of a property, level, or other parameter by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

The term “pharmaceutically acceptable” can refer to compounds and compositions which can be administered to a subject (e.g., a mammal or a human) without undue toxicity.

As used herein, the term “pharmaceutically acceptable carrier” can include any material or substance that, when combined with an active ingredient allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. The term “pharmaceutically acceptable carriers” excludes tissue culture media.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

It should be understood that this invention is not limited to the particular methodologies, protocols, and reagents, etc., described herein and as such can vary therefrom. The terminology used herein is for the purpose of describing particular embodiments only, and is not intended to limit the scope of the present invention, which is defined solely by the claims.

### Suicide

Suicide is the act of intentionally taking one's own life. In the United States, 41,149 took their lives in 2013, the most recent year for which full data are available. Suicide accounted for 12.6 deaths for every 100,000 people nationwide; making it the country's 10th leading cause of death. Unlike many other leading causes of death, suicide continues to claim more lives each year. In that same year, 494,169 people visited a hospital for injuries due to self-harm behavior, suggesting that approximately 12 people harm themselves (not necessarily intending to take their lives) for every reported death by suicide. Together, those harming themselves made an estimated total of more than 650,000 hospital visits related to injuries sustained in one or more separate incidents of self-harm behavior.

Suicide does not discriminate and can affect people of any gender, age or ethnicity. While the risk for suicidal behavior is complex, the people of highest risk include those suffering from mental disorders (e.g., depression, anxiety, bipolar disorder, schizophrenia), substance abuse, a history of abuse, a prior suicide attempt, or those that have a family history of suicide, a family history of mental disorders or substance abuse, are incarcerated, or have access to firearms or drugs in the home. The accumulation of long-term stresses and/or short term stress or trauma can increase feelings or desire for suicide in an individual.

At present there are few preventative measures for an individual to use during a period of suicidality. Current treatments/preventative measures typically include some type of psychotherapy (e.g., talk therapy), such as cognitive-behavioral therapy (CBT) or dialectical behavior therapy (DBT).

Suicidal ideation and behavior can be assessed using the Columbia-Suicide Severity Rating Scale (C-SSRS), and comprises ten distinct categories as follows:

| | |
|---|---|
| Category 1 | Wish to be Dead |
| Category 2 | Non-specific Active Suicidal Thoughts |
| Category 3 | Active Suicidal Ideation with Any Methods |
| | (Not plan) without Intent to Act |
| Category 4 | Active Suicidal Ideation with Some Intent to |
| | Act, without Specific Plan |
| Category 5 | Active Suicidal Ideation with Specific Plan and |
| | Intent |
| Category 6 | Preparatory Acts or Behavior |
| Category 7 | Aborted Attempt |
| Category 8 | Interrupted Attempt |
| Category 9 | Actual Attempt (non-fatal) |
| Category 10 | Completed Suicide |

Each of the above categories has a binary response (yes/no). In addition the C-SSRS has an additional outcome that is not suicide related, which includes “self-injurious behavior without suicidal intent” that also carries a binary response (yes/no).

The C-SSRS questionnaire or another scale to determine the subject's risk of suicide can be used by a health care professional to determine if the subject should be provided or prescribed a xenon composition, for example, in a self-administration device.

While an individual need not be familiar with the C-SSRS to administer a xenon composition either to themselves or another, it will be understood that the intent of administration of a xenon composition will be to move a subject backwards from a higher number category (e.g., category 9) to a lower number category (e.g., category 1). In some embodiments, administration of a xenon composition can move the individual off of the scale entirely such that they are below a category 1, i.e., no longer wish to be dead.

A second individual, such as an emergency responder or healthcare professional, can readily assess and/or approximate the category that a subject is currently experiencing by asking a few simple questions. For example, an individual can ask the subject “Are you feeling suicidal?” to which an answer of ‘yes’ would put the subject onto the C-SSRS chart at at least a category 2. Similarly, “Do you have a plan?” would indicate whether a subject is above or below a category 5. Finally, “Do you have the means?” (e.g., a gun, access to drugs etc.) would indicate if the subject is above or below a category 6.

It is also contemplated herein that a family member or friend can administer xenon to a subject, if they should interrupt a suicide attempt by the subject (e.g., a category 8 on the C-SSRS scale).

In addition, it is further contemplated herein that each of categories 1-5 can be further broken down into subcategories, which can reflect e.g., the intensity the thoughts/feeling experienced by a subject in each category. For example, a subject can be a “Category 5—Low” in which the subject has formulated a plan and has some intent but is not at the point where they are considering a move into “Category 6.” In this example, a subject can be a “Category 5—High” if the subject has formulated a plan, has intent and is making a mental plan for the preparatory actions of Category 6. In some embodiments, administration of a xenon composition may not move a subject down an entire category but can move them to a lower subcategory within each category.

In some embodiments, administration of the xenon composition is repeated until the urge to suicide has lessened or passed.

### Self-Harm or Deliberate Self-Harm

Self-harm or deliberate self-harm is often defined as the intentional injuring of body tissue, generally without suicidal intent, and includes both self-injury and self-poisoning. The most common form of self-harm is skin-cutting, but self-harm also covers a wide range of behaviors including, but not limited to, burning, scratching, banging or hitting body parts, interfering with wound healing (e.g., dermatillomania), hair-pulling (e.g., trichotillomania) and the ingestion of toxic substances or objects. Generally suicide is not the intent or goal of self-harming behaviors, however self-harm can be potentially life-threatening. While self-harm can occur at any age, it is particularly common in adolescents and during young adulthood, typically emerging between the ages of 12 and 24.

The motivations for self-harm vary and it can serve many functions, including acting as a coping mechanism which provides temporary relief of intense feelings, such as anxiety, depression, stress, emotional numbness, a sense of failure, or self-loathing. Self-harm can also be a way for an individual to deal with mental traits including low self-esteem or perfectionism. Self-harm is often associated with a history of trauma and abuse, including emotional and sexual abuse. There are a number of different methods that can be used to treat self-harming behaviors, which concentrate on either treating the underlying causes or on treating the behavior itself. When self-harm is associated with depression, antidepressant drugs and treatments may be effective. Other approaches involve avoidance techniques, which focus on keeping the individual occupied with other activities, or replacing the act of self-harm with safer methods that do not lead to permanent damage.

There are a number of scales that can be used to assess a subject's risk of self-harming behaviors and/or the degree of such behaviors. These scales or questionnaires can be used by a friend, relative, emergency responder, or healthcare professional to determine if a subject should be administered xenon to reduce the intensity of such self-harming thoughts and/or feelings and/or behaviors. Alternatively, these questionnaires can be used by a health care professional to determine if a xenon composition should be prescribed such that the xenon can be self-administered during periods when the subject has an uncontrollable urge to self-harm, such as when the subject is under distress. Examples of useful scales or questionnaires relating to self-harming behaviors include, e.g., Chronic Self-Destructiveness Scale (CSDS), Self-Harm Behavior Study, Self-Injury Survey, Impulsive and Self-Harm Questionnaire, Self-Injurious Behavior Questionnaire (SIB-Q), Self-Injury Questionnaire (SIQ), Timed Self-Injurious Behavior Scale, Deliberate Self-Harm Inventory (DSHI), Adolescent Risk Inventory and the Self-Harm Inventory (SHI).

A subject need not be familiar with any of the above-mentioned scales in order to self-administer xenon. The subject can simply administer xenon, e.g., whenever they deem the urge to self-harm is uncomfortable or they feel that they are losing control of the ability to prevent their self-harming behaviors. Typically, administration (e.g., self-administration, or administration by another) of the xenon composition is repeated until the urge to self-harm has lessened or passed as noted by the subject to which the xenon was administered.

### Homicidal Ideation

“Homicide” is generally defined as the act of one human being causing the death of another human being, while “homicidal ideation” refers to thoughts about homicide. Homicidal ideation is noted to be an important risk factor when assessing a person's risk for violence. This type of assessment is routine for psychiatric patients or any other patients presenting to hospital with mental health complaints. There are many associated risk factors which include: history of violence and thoughts of committing harm to another, poor impulse control and an inability to delay gratification, impairment or loss of reality testing, especially with delusional beliefs or command hallucinations, the feeling of being controlled by an outside force, the belief that other people wish to harm him/her, the perception of rejection or humiliation at the hands of others, being under the influence of substances or a past history of antisocial personality disorder, frontal lobe dysfunction or head injury.

The Centers for Disease Control and Prevention (CDC) provide a Danger Assessment test that can be used to assess a subject's potential assault and homicidal danger level.

| | Immediate | |
|---|---|---|
| Key to | Dangerousness | |
| Danger | to Others | Typical Indicators |
| 1 | No | Has no assaultive or homicidal |
| | predictable | ideation, urges, or history of same; |
| | risk of | basically satisfactory support system; |
| | assault or | social drinker only |
| | homicide | |
| 2 | Low risk | Has occasional assault or homicidal |
| | of assault | ideation (including paranoid ideas) |
| | or homicide | with some urges to kill; no history of |
| | | impulsive acts or homicidal attempts; |
| | | occasional drinking bouts and angry |
| | | verbal outbursts; basically satisfactory |
| | | support system. |
| 3 | Moderate | Has frequent homicidal ideation and |
| | risk of | urges to kill but no specific plan; |
| | assault | history of impulsive acting out and |
| | or homicide | verbal outbursts while drinking, on |
| | | other drugs, or otherwise; stormy |
| | | relationship with significant others with |
| | | periodic high-tension arguments. |
| 4 | High risk | Has homicidal plan; obtainable means; |
| | of homicide | history of substance abuse; frequent |
| | | acting out against others, but no |
| | | homicide attempts; stormy |
| | | relationships and much verbal fighting |
| | | with significant others, with occasional |
| | | assaults. |
| 5 | Very high | Has current high-lethal plan; available |
| | risk of | means; history of homicide attempts or |
| | homicide | impulsive acting out, plus feels a strong |
| | | urge to control and “get even” with a |
| | | significant other; history of serious |
| | | substance abuse; also with possible |
| | | high-lethal suicide risk. |

Typically xenon is administered to move a person from a high or very high risk of homicide, back to a lower risk of suicide. It is contemplated herein that a subject with homicidal ideation may not be compliant with self-administration of xenon, thus in some embodiments, the subject being treated for homicidal ideation or for having a high risk of homicide will be administered xenon by a health professional or emergency responder. It is further contemplated herein that xenon can be administered into a hostage situation (e.g., into a ventilation source) in order to de-escalate the risk of homicide, suicide or both.

In one embodiment, the homicide comprises a murder-suicide.

Detection and/or Quantification of Biomarkers

In some embodiments, a subject is first selected for treatment with a xenon composition by the detection of at least one biomarker in a biological sample obtained from a subject. The term “biological sample” as used herein refers to a cell or population of cells or a quantity of tissue or fluid from a subject. Most often, the sample has been removed from a subject, but the term “biological sample” can also refer to cells or tissue analyzed in vivo, i.e., without removal from the subject. Biological samples include, but are not limited to, whole blood, plasma, serum, urine, saliva, tissue biopsy, cell culture, or cerebrospinal fluid. A biological sample or tissue sample can also refer to a sample of tissue or fluid isolated from an individual, including but not limited to, for example, blood, plasma, serum, urine, stool, sputum, spinal fluid, pleural fluid, nipple aspirates, lymph fluid, the external sections of the skin, respiratory, intestinal, and genitourinary tracts, tears, saliva, milk, circulating cells (including but not limited to blood cells), tumors, organs, and also samples of in vitro cell culture constituent. The term “sample” includes any material derived by processing such a sample. Derived samples may, for example, include nucleic acids or proteins extracted from the sample or obtained by subjecting the sample to techniques such as amplification or reverse transcription of mRNA, isolation and/or purification of certain components, etc. In general, the means for obtaining a sample from a subject will be minimally invasive (e.g., blood test).

Exemplary biomarkers to aid in the assessment of the risk of a suicidal, homicidal or self-harming episode include, but are not limited to, quinolinic acid, kynurenine, platelet 5HT₂ₐ (serotonergic) receptors, and cerebrospinal fluid 5-hydroxyindoleacetic acid (SHIAA).

Methods to measure gene expression products (e.g., proteins, RNA etc.) associated with the biomarkers described herein are well known to a skilled artisan. Such methods to measure gene expression products, e.g., protein level, include ELISA (enzyme linked immunosorbent assay), Western blot, and immunoprecipitation, immunofluorescence using detection reagents such as an antibody or protein binding agents. Alternatively, a peptide can be detected in a subject by introducing into a subject a labeled anti-peptide antibody and other types of detection agent. For example, the antibody can be labeled with a radioactive marker whose presence and location in the subject is detected by standard imaging techniques. The expression of nucleic acids can be measured using e.g., PCR procedures, RT-PCR, Northern blot analysis, differential gene expression, RNA protection assay, microarray analysis, hybridization methods, next-generation sequencing etc. Non-limiting examples of next-generation sequencing technologies can include Ion Torrent, Illumina, SOLiD, 454; Massively Parallel Signature Sequencing solid-phase, reversible dye-terminator sequencing; and DNA nanoball sequencing.

In some embodiments, assays for one or more of the biomarkers described herein can be obtained commercially from e.g., SB DRUG DISCOVERY, SIGMA ALDRICH, MYBIOSOURCE.COM, USCN BUSINESS CO. LTD, CLOUD CLONE CORP., LIFESPAN BIOSCIENCES, BIOMATIK, ALPCO, ABBEXA LTD, and ROCKY MOUNTAIN DIAGNOSTICS.

The results obtained for the expression and/or activity of at least one biomarker from a biological sample can be compared and/or normalized to a reference. As used herein, the term “reference value ” refers to a reference value, or range of values, obtained for one or more markers as described herein from e.g., at least one subject. In some embodiments, the reference value is obtained from the same subject prior to onset of a suicidal, homicidal or self-harming episode or from a population of subjects that are substantially free of such thoughts/behaviors. Alternatively, the reference value or range of values can be obtained from a subject or plurality of subjects determined to have a suicidal, homicidal, or self-harming episode at the time the biological sample was obtained. The reference sample can be stored as a value(s) on a computer or PDA device to permit comparison with a value obtained from a subject using the methods described herein. The reference sample can also be obtained from the same subject e.g., at an earlier time point prior to onset of the thoughts/behaviors described herein or prior to initiation of treatment with a xenon composition using clinical tests known to those of skill in the art. One of skill in the art can determine an appropriate reference sample for use with the methods described herein.

In one embodiment, a biological standard is obtained at an earlier time point (e.g., prior to treatment with a xenon composition) from the same individual that is to be tested or treated as described herein. Alternatively, a standard can be from the same individual having been taken at a time after the treatment with a xenon composition. In such instances, detection and/or quantification of one or more biomarkers can provide a measure of the efficacy of treatment of the xenon composition.

Generally, an increase in the amount of expression over a reference value (e.g., a reference obtained from the subject prior to onset of a suicidal, homicidal or self-harming episode) indicates that the subject is at an increased risk of suicidal, homicidal or self-harming episodes when using a reference value from a subject or population of subjects that are not currently experiencing a suicidal, homicidal or self-harming episode. As will be apparent to one skilled in the art, when using a reference value obtained from a subject or population of subjects that were experiencing a suicidal, homicidal or self-harming episode, a biomarker level at or above the level of the reference sample would be considered a high-risk subject while the biomarker level below the level of the reference sample would be considered low risk.

### Xenon Compositions

Xenon can be self-administered via a gas-inhalation apparatus or can be administered by a second individual, such as a relative, friend, co-worker, healthcare professional (e.g., psychiatrist) or emergency responder (e.g., EMT, firefighter, police officer, trauma center staff, doctor or nurse). It is contemplated herein that medical-grade xenon is packaged in cylinders or canisters at specified concentrations (% xenon in oxygen) that would administer a metered unit dose to prevent the possibility of asphyxiation or deep sedation. In some embodiments, the xenon-containing compositions are formulated for self-administration by the subject. For example, xenon compositions can be administered using a hand-held canister or device packaged for self-administration by the individual.

The xenon compositions are typically formulated as a gas but can also be a formulation comprising a nanoparticle (e.g., liposome) or a nanosponge. In certain embodiments, xenon gas administered to a subject (e.g., via inhalation) comprises between 0.5% to 35% xenon by volume, 1% to 35% xenon, 2% to 35% xenon, 5% to 35% xenon by volume, e.g., 0.5%-1%, 0.5%-5%, 0.5%-10%, 0.5%-15%, 1%-2%, 1%-5%, 1%-10%, 1%-15%, 1%-20%, 1%-25%, 1%-30%, 1%-35%, 1.5%-5%, 1.5%-10%, 1.5%-15%, 1.5%-20%, 1.5%-30%, 1.5%-35%, 2%-5%, 2%-10%, 2%-20%, 2%-30% 10%-35%, 10% to 30%, 10% to 20%, or 20% to 30% xenon by volume.

Xenon gas administered to the subject can be balanced in part with oxygen gas (O₂) (e.g., 20% to 35% by volume, 20% to 30% by volume, or 20% to 25% by volume), nitrogen gas (N₂) (e.g., 25% to 75% by volume, 25% to 40% by volume, 30% to 70% by volume, 30% to 60% by volume, or 30% to 50% by volume). In some embodiments, the xenon composition as described herein comprises 30% xenon by volume, 30% oxygen (O₂) by volume, and 40% nitrogen (N₂) by volume or contains 30% xenon by volume, 21% oxygen by volume, and 49% nitrogen by volume. Xenon gas compositions can also contain vaporized water (e.g., be humidified). In one embodiment, the concentration of oxygen is not lower than 21% by volume.

In some embodiments, the xenon composition(s) used in the methods described herein, can also contain one or more additional pharmaceutical agents or additional therapeutic agents (e.g., aerosolized therapeutic agents) for treating a psychiatric disorder (e.g., anxiety, depression, psychosis) or decreasing one or more of the symptoms of a psychiatric disorder as disclosed herein. It is contemplated herein that the additional therapeutic agents are those that can produce an immediate effect without requiring sustained or daily dosing for e.g., 6-8 weeks to achieve an effect. Some exemplary antidepressants or anti-anxiety medications that are fast acting and can be formulated for aerosol delivery include, but are not limited to, benzodiazepines (e.g., lorazepam, diazepam, alprazolam, bromazepam, clonazepam etc.), ketamine, scopolamine, MI-4, beta blockers (e.g., propranolol, atenolol, etc.), among others. Exemplary anti-psychotics that can be used in combination with xenon include, but are not limited to, haloperidol, ziprasidone, olanzapine, loxapine, molindone, fluphenazine, risperidone, etc.

The xenon compositions as described herein can also be formulated as liquids containing xenon gas in echogenic liposomes (e.g., 10% to 100% saturation of xenon gas, 20% to 100% saturation of xenon gas, 40% to100% saturation of xenon gas, 50% to 100% saturation of xenon gas, 60% to 100% saturation of xenon gas, 20% to 80% saturation of xenon argon gas, 40% to 80% saturation of xenon gas, or 60% to 80% saturation of xenon gas). It is specifically contemplated herein that such caged formulations of xenon are administered at regular intervals and xenon is activated or released from the liposomes using a stimulus, such as ultrasound waves. While not required, such methods for administering xenon are typically done in a clinical setting and such methods are not considered for self-administration.

A single dosage of a liquid containing liposomes can be at least 20 mL to 200 mL, or 20 mL to 100 mL. In one embodiment, 1 mL of liposomes can contain 1.8 mL xenon gas. Liposomes that can be used in any of the methods described herein can contain a phospholipid bilayer and a core (e.g., a core of a xenon saturated liquid or a core of xenon gas). Non-limiting examples of such liposomes, as well as methods of their preparation, are described in Britton et al. (Circulation 122:1578-1587, 2010) and Cullis et al. (Advanced Drug Delivery Reviews 3:267-282, 1989, and all references cited therein).

### Modes of Administration

As described herein, the xenon compositions (e.g., a xenon gas) can be self-administered by the subject or alternatively, administered by another individual. In one embodiment, the xenon gas is not administered during a psychotherapy session. A unit dose (e.g., a net single exposure to a xenon composition) can be between 0.5% to 35% xenon or between 10% to 35% xenon, a preferred unit dose can be between 10% to 30% xenon, such as 20% to 25% xenon. For example, administration of 35% xenon by inhalation for 1 hour at a normal ventilation rate of 6 liters/minute requires 360 liters of 35% xenon or 126 liters of 100% xenon.

In other embodiments, a single dose or unit dose (e.g., a net single exposure to xenon) can be between 0.5% to 35%, for example, 0.5% to 5%, 0.5% to 10%, 0.5% to 15%, 0.5% to 20%, 0.5% to 25%, 0.5% to 30%, 1.5% to 5%, 1.5% to 10%, 1.5% to 15%, 1.5% to 20%, 1.5% to 25%, 1.5% to 30%, 30% to 35%, 25% to 35%, 20% to 35%, 10% to 35%, 5% to 35%, 1% to 35%, 15% to 25%, 10% to 25%, 15% to 20%, 10% to 20%, 5% to 25%, 5% to 20%, or any range in between. The total amount of xenon required for this therapy may be less as xenon recovery systems can be used that allow for xenon recycling (e.g., Rawat and Dingley, Anesthesia and Analgesia 110:101-109, 2010).

The xenon composition in the form of a gas can be administered to the subject in a variety of ways, including, but not limited to, by inhalation, intraocularly, or intranasally, and such administration can provide a therapeutic effect as xenon gas is capable of crossing the blood/brain barrier in a subject. For example, a single administration a xenon composition (e.g., any of those described herein) can be administered over a continuous time period (e.g., 5 min, 10 min, 15 min, 30 min, 1 h, 2 h, 3 h, 4 h, 5 h, 12 h, 24 h etc.) or can be administered in discrete doses (e.g., a single metered dose, or a repeated metered dose). In certain embodiments, the xenon gas is administered or self-administered ad libitum until the subject experiences a decrease or relief of at least one symptom associated with suicide, homicide or self-harm. In some examples of the methods described herein, a xenon gas is administered as a bolus (e.g., by inhalation of xenon gas).

Liquid and solid xenon compositions (e.g., nanoparticles or nanosponges) can also be administered to a subject by many routes of administration, including, but not limited to, intravenously, intraarterially, subcutaneously, intranasally, or intraocularly. It is specifically contemplated herein that xenon is administered in a protected or caged formulation (e.g., in a nanoparticle) and can be rapidly released using a stimulus for the acute treatment of thoughts and/or feelings of suicide, homicide or self-harm. Typically, this mode of administration would occur in a clinical setting under the guidance of a healthcare professional or one of skill in the art.

A nanoparticle (e.g., liposome) or nanosponge (e.g., any of the delivery systems described herein) containing xenon gas can be administered to a subject one or more (e.g., two, three, or four) times before or during an acute episode of suicidal, self-harming or homicidal feelings, or over a continuous time period (e.g., 5 minutes, 10 minutes, 15 minutes, 20 minutes, 25 minutes, 30 minutes, 40 minutes, 1 hour, 2 hours, 3 hours, 4 hours, 5 hours, 6 hours, 7 hours, 8 hours, 9 hours, 10 hours, 11 hours, 12 hours, 1 day, 2 days, or 3 days, or overnight (e.g., 6 to 13 hours)) during an acute episode of suicidal, self-harming or homicidal feelings, or can be administered as a bolus (e.g., as a liquid to be injected or administered to the eye) during such acute episodes. Administration of a caged format of xenon, such as a nanoparticle or nanosponge can require a stimulus to release the xenon gas from the drug delivery device. In some embodiments of the methods described herein, echogenic waves are used to disrupt the nanoparticles (e.g. echogenic liposomes) or nanosponges containing xenon gas in one or more tissues (e.g., brain tissue or spinal cord) in the subject.

During a prolonged period of suicidal, homicidal or self-harming thoughts and/or feelings, a xenon composition can be administered to the subject once a day, twice a day, three times a day, four times a day, once a week, twice a week, three times a week, four times a week, five times a week, six times a week, seven times a week, once every two weeks, twice every two weeks, three times every two week, four times every two weeks, once a month, twice a month, three times a month, four times a month, five times a month, six times a month, seven times a month, or 8 times a month). As described herein, each administration can take place over a continuous period of time or the administration can be provided as a bolus. Alternatively, it is specifically contemplated herein that the subject can self-administer a xenon composition at their own discretion e.g., ad libitum and/or to achieve partial or complete remission of at least one symptom of suicidal, homicidal or self-harming thoughts and/or feelings.

In any of the methods described herein, the frequency, duration, and or dosage of administration of a xenon composition can be dependent on the occurrence of the episodes of suicidal, homicidal or self-harming thoughts and/or feelings. Such variation of the frequency, duration, and/or dosage of single administrations of the xenon composition can be performed by the subject themselves, a friend, a coworker, an emergency responder or a health care professional based on the subject's thoughts and/or feelings and/or communications between the subject and another individual. For example, an emergency responder or health care professional can alter the frequency, duration, and/or dosage of single administrations of the xenon composition during an episode of suicidal, homicidal or self-harming thoughts and/or feelings following the assessment of the severity, frequency, or duration of one or more symptoms of such an episode in the subject. That is, the route, frequency of duration and or dosage of administration can be varied by the subject themselves, another individual or one of skill in the art. Individual administrations do not have to take place over a common continuous time period. For example, at least one single administration can take place over a period of 1 hour and at least one additional administration can take place over a period of 8 hours. For example, 35% xenon administered as inhalation therapy at normal breathing rates for 1 hour (360 liters total volume at a minute volume of 6 liters/min for a healthy adult) would require 126 liters of xenon. Lower xenon doses (e.g., 10%) would require 36 liters per hour of inhalation therapy. This xenon can be recovered and reused (e.g., Rawat and Dingley, Anesthesia and Analgesia 110:101-109, 2010), so it is possible that the total amount of xenon to support an hour of inhalational therapy can be less than 126 or 36 liters.

### Dosage and Administration

A “therapeutically effective amount” or “therapeutically effective dose” of a xenon composition is that amount that, when administered results in an improved therapeutic benefit relative to that observed in the absence of administering the xenon composition. For example, a therapeutically effective dose or amount of a xenon and/or argon composition is the amount of xenon that reduces NMDA receptor activation or transmission in the brain relative to the level of NMDA receptor activation or transmission in the brain in the absence of administration of the xenon. As another example, a therapeutically effective dose or amount of a xenon composition is the amount of xenon that affects activity of other xenon targets including, for example, cholinergic receptors, 5-HT3 receptors, TREK channels, KATP channels, among others. Typically, the therapeutically effective amount of a xenon composition is an amount that can reduce the intensity of feelings and/or thoughts of suicide, self-harm and/or homicide. In such embodiments, the intensity of such feelings and/or thoughts is reduced by at least 20%; in other embodiments, administration of the xenon composition reduces the intensity of suicide, self-harming, or homicide thoughts and/or feelings by at least 30%, at least 40%, at least 50%, at least 60%, at least 70%, at least 80%, at least 90%, at least 95%, at least 99%, or even 100% (i.e., eliminating such thoughts and/or feelings completely during the time xenon is being administered). In some embodiments, the subject can self-report the intensity of such suicidal, homicidal or self-harming episodes to themselves or another individual, for example, using an arbitrary scale of 1 to 10, where 10 represents a strong desire for suicide, self-harm or homicide or even an attempted suicide, self-harming episode or homicide, while 1 represents little to no desire for suicide, self-harm or homicide. In such embodiments, xenon can be administered repeatedly or continuously until the subject moves to a number (e.g., 4) on such a scale that represents a tolerable intensity of such feelings of desire for suicide, self-harm or homicide, e.g., an intensity that can be tolerated by the individual without a need to suicide, homicide or self-harm. It is important to note that this tolerable level will be subject and/or situation specific. For example, some individuals may find a tolerable intensity of suicidal feelings at a “4,” while others may find a “6” to be tolerable, in that they have the feelings but are not compelled to act on them. An individual under a lot of stress or recently suffering a loss may find they are less tolerant to suicidal, homicidal or self-harming feelings and may need to treat with xenon until a level of e.g., “2” is achieved in order to feel as though they can tolerate the feelings without acting on them. In other embodiments, an accepted scale such as the Columbia Suicide Severity Rating Scale, the CDC's Danger Assessment test, or a clinically accepted self-harming scale are used to assess the intensity of such sensations of suicide, homicide or self-harm in order to modify the dose accordingly.

The therapeutically effective dose of a xenon composition can be administered using any medically acceptable mode of administration. Although the skilled artisan would contemplate any of the modes of administration known to one of ordinary skill, preferably xenon is administered by inhalation so that the effect will have a rapid onset and can be easily and rapidly titrated for appropriate treatment.

Therapeutic compositions of the agents disclosed herein can include a physiologically tolerable carrier together with xenon gas as described herein, dissolved or dispersed therein as an active ingredient. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without toxicity or the production of undesirable physiological effects such as nausea, dizziness, gastric upset and the like. A pharmaceutically acceptable carrier will not itself promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as topical agents or injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein.

Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

In some embodiments, it can be advantageous to formulate the aforementioned pharmaceutical compositions in dosage unit form for ease of administration and uniformity of dosage. Dosage unit or unitary form refers to physically discrete units suitable as unitary dosages (e.g., a metered inhaled dose), each unit containing a predetermined quantity of xenon gas calculated to produce the desired therapeutic effect in association with the required pharmaceutical carrier.

### Efficacy

The efficacy of a composition comprising xenon for the treatment and/or prevention of suicide, homicide and self-harming behavior can be self-reported by the subject, or determined by the skilled clinician. However, a treatment is considered “effective treatment,” as the term is used herein, if any one or all of the signs or symptoms of, as but one example, suicidality, homicidality or self-harming behavior are altered in a beneficial manner, or the clinically accepted symptoms or markers of disease are improved or ameliorated, e.g., by at least 10% following treatment with a xenon composition. Efficacy can also be measured by failure of an individual to worsen as assessed by hospitalization or need for medical interventions (e.g., first aid for wounds, inpatient care to monitor suicidality). Efficacy in a population of patients can also be determined by measuring mortality rates due to suicide or homicide. Methods of measuring these indicators are known to those of skill in the art and/or described herein.

Typically treatment is considered successful when the episode of suicidality, homicidality or self-harming behavior has passed or is relieved in intensity. Successful treatment does not require that future episodes of suicidal, homicidality or self-harming behavior be prevented, alleviated or eliminated. Treatment with a xenon composition is considered efficacious if a subject moves backwards on a clinically acceptable scale of risk of suicide, homicide, or self-harming behavior or if the intensity of the thoughts and/or feelings of suicide, homicide, or self-harming behavior are reduced by at least 20% as reported by the subject. In some embodiments, treatment is successful if a suicide, homicide or self-harming episode does not end in an act of suicide, homicide or self-harm.

Xenon has also been shown to act on hyperpolarization-activated cyclic nucleotide cation (HCN) channels (Mattusch et al., Anesthesiology 2015), which can modulate thalamocortical signaling. Without wishing to be bound by theory, the action of xenon on HCN channels could have implications for disrupting abnormal thought patterns that exist in states of mental crises. Thus, in certain embodiments, the activity of HCN channels can be used to test the efficacy of a xenon composition in a clinical setting.

### Xenon Composition Delivery Systems

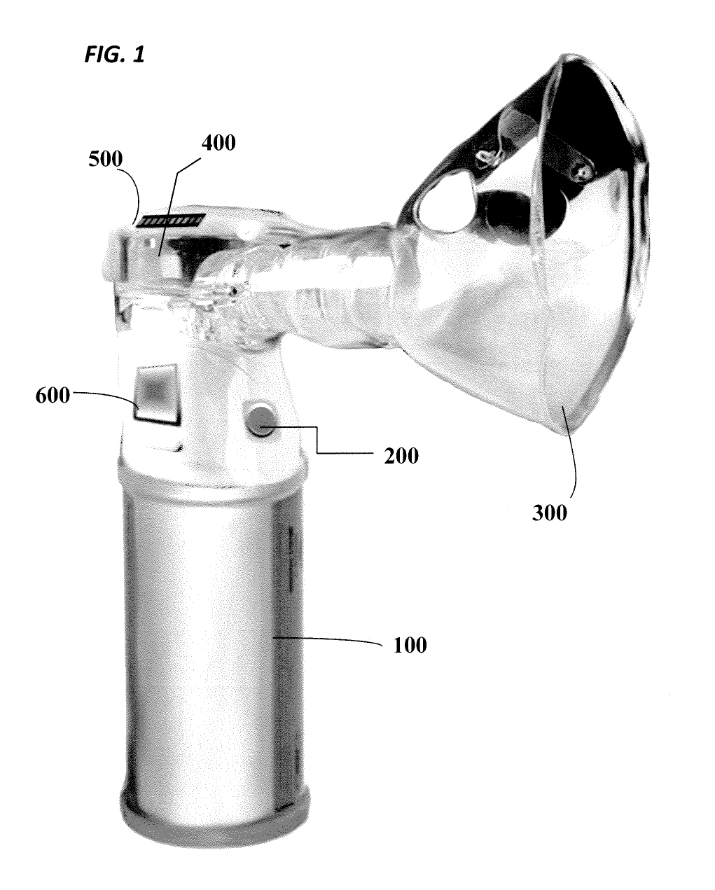

A xenon gas (e.g., any of the xenon gas compositions described herein) can be administered to the subject through the use of a self-contained breathing apparatus (e.g., a mouthpiece, face mask, or other hand-held device, a continuous positive airway pressure (CPAP) device, and/or a nose mask) or a nebulizer (inhaler). FIG. 1 shows a non-limiting example of a face mask and a portable xenon gas canister that can be used in the methods described herein. FIG. 1 is a diagrammatic representation of a xenon self-administration device that can be used for on-demand inhalation of a therapeutic xenon gas. The self-administration device comprises a replaceable canister 100 comprising a therapeutic xenon gas (e.g., 5-35% xenon gas in air, for example 30% xenon/air) with a push button 200 to activate gas flow to the mask 300. The device further comprises a built-in regulator 400 that transforms high canister pressure to a breathable level (e.g., ˜50 psi) and a graded flowmeter readout 500 that indicates when gas is flowing and/or indicates the amount of gas remaining in the canister. In one embodiment, the device further comprises a medication event monitoring system (MEMS) 600 that can be activated with device use (e.g., can report initial use, a threshold number of uses over a designated time period, or a low gas warning). Such medication event monitoring systems can be connected to a computer by wireless technology, including e.g., Wi-Fi or Bluetooth™ technologies. The MEMS system can be used to alert a friend, co-worker, healthcare professional (e.g., doctor, psychiatrist etc.) or directly to a 911 or other emergency service. The device can further comprise additional safety features, including the ability to delay a subsequent dose of the composition for a desired period of time, keypads to unlock the dosing feature so that the self-administration device can be unlocked by the designated user only, auto-shut off after a desired length of time of continuous administration, warning lights or alarms to indicate a regulator malfunction, anti-tip features to ensure the canister stands safely on a flat surface and will not fall or roll off of e.g., a table or counter, among others.

Non-limiting examples of mouthpieces or face masks that can be used to administer a xenon gas are described in U.S. Pat. Nos. 6,125,844; 6,247,470; 6,981,502; 7,870,860; 7,775,208; 7,000,611; 6,981,502; 6,779,521; 7,866,320; 6,779,521; 5,413,095; 6,408,853; 5,348,000; 6,715,485; 5,243,971; and 6,112,746; and U.S. Patent Application Nos. 2005/0205098, 2008/0223369; 2004/030639 (each of the listed patents and patent application publications are herein incorporated by reference in their entirety). Non-limiting examples of nose masks that can be used to administer xenon gas are described in U.S. Pat. Nos. 4,354,488; 7,207,333; 6,439,235; 6,807,966; 4,660,555; 4,742,824; 6,595,215; and 4,721,060, and U.S. Patent Application Publication Nos. 2010/0242959 and 2004/030639 (each of the listed patents and patent application publications are herein incorporated by reference in their entirety). When using such masks, typical dosages of xenon gas range from 0.25 to 1.0 liters per hour of any of the xenon gas compositions described herein.

Xenon gas compositions can also be administered to the subject intraocularly, for example, through the use of self-contained goggles (e.g., goggles that can be worn one or more hours (e.g., at least two, three, four, five, or six hours) or overnight (e.g., between 5-13 hours). The goggles can also be equipped with a liquid crystal display (LCD) screen and/or audio speakers (which allow the subject to watch a movie or images and/or listen to sound while wearing the goggles). A tank (e.g., a portable tank) containing a xenon gas composition can be provided for use with the self-contained breathing apparatus or the self-contained goggles. For example, a system for performing the methods described herein can contain sealable goggles that cover a subject's eyes that contain at least one opening that allows xenon gas to enter the space enclosed by the goggles and a source of xenon gas (e.g., a tank of xenon gas), where the sealable goggles and the xenon gas are connected to each other. The systems provided can further include tubing that connects the sealable goggles to the source of xenon gas. The sealable goggles can further contain a strap that allows the sealable goggles to be positioned (e.g., securely positioned) over the subject's eyes. When using such goggles, the xenon gas composition can contain up to 100% xenon, with the rest of the gas being either air or comprising a mixture of O₂ and N₂ (as described herein). Typical application pressures of a xenon gas composition administered to the eyes range from 5 to 10 mbar, e.g., 5 to 10 mbar, which results in a blood level of xenon of about 100 to 200 nL xenon/mL blood. A dose that may be administered to the subject using the goggles is 5-10 mbar xenon gas per one hour.

In any of the methods described herein, a subject can be further administered one or more additional pharmaceutical agents or therapeutic agents for treating an acute episode of suicidal, homicidal of self-harming feelings or for decreasing the severity of one or more symptoms associated with suicidality, self-harm or homicidality. Specifically contemplated herein are pharmacological agents useful for treating common psychiatric disorders that can be a risk factor for suicidal, self-harming or homicidal thoughts and/or feelings. Typically, the drugs to be administered in combination with xenon will be those drugs that can be administered in an inhaled form and/or can produce a rapid on/off effect. In some embodiments, an inhaled antidepressant, such as ketamine, can be administered in combination with xenon.

A xenon administration device, particularly a self-administration device can also comprise a number of safety features including, for example, controlled intervals between doses of xenon to prevent accumulation of the gas or a secondary drug in the subject's system. That is, once the device is used to administer a dose, it is not able to be activated for a second dose until a desired time interval has passed. This is particularly relevant when the administration device also administers a second drug, such as an inhaled antidepressant since xenon has a rapid ‘on/off’ effect and it is unlikely to accumulate in the subject's system.

The present invention may be as defined in any of the following numbered paragraphs:

1. A method for reducing the intensity of thoughts and/or feelings associated with suicide, self-harm, or homicide, the method comprising: administering a xenon composition to a subject in need thereof, thereby reducing the intensity of the thoughts and/or feelings.

2. The method of paragraph 1, wherein the xenon composition is self-administered.

3. The method of paragraph 1 or 2, wherein the xenon composition is administered by a second individual.

4. The method of paragraph 3, wherein the second individual comprises an emergency responder, a friend, a relative, a co-worker, or a health care professional.

5. The method of any one of paragraphs 1-4, wherein the xenon composition comprises at least 0.5-35% xenon by volume.

6. The method of any one of paragraphs 1-5, wherein the xenon composition further comprises air, nitrogen, oxygen, or a combination thereof.

7. The method of any one of paragraphs 1-6, wherein the administration of xenon is a single exposure to xenon.

8. The method of any one of paragraphs 1-7, wherein the administration of xenon is repeated as necessary until the intensity of the thoughts and/or feelings is reduced by at least 20%.

9. The method of any one of paragraphs 1-8, wherein the administration of xenon is repeated as necessary until the intensity of the thoughts and/or feelings are reduced to a tolerable level.

10. The method of paragraph 9, wherein the tolerable level is a level where the subject feels able to control or prevent self-harming or homicidal actions.

11. The method of any one of paragraphs 1-10, wherein the xenon composition is administered by inhalation.

12. The method of any one of paragraphs 1-11, wherein the xenon composition is not administered during a psychotherapy session.

13. A device for self-administration of xenon, the device comprising: (a) a canister containing a xenon gas stored at a high-pressure level; (b) a regulator in which the xenon gas is transformed from the high-pressure level to a breathable level; (c) a face mask communicatively coupled to the canister and the regulator and through which the xenon composition is externally administered, the face mask being connected to the canister and the regulator such that the xenon gas flows from the canister, through the regulator, and into the face mask; and (d) a medication event monitoring system communicatively coupled with the canister and configured to detect and/or determine changes in the xenon gas.

14. The device of paragraph 13, wherein the device further comprises a push button configured to activate xenon gas flow from the canister to the face mask.

15. The device of paragraph 13 or 14, further comprising a graded flowmeter readout indicative of xenon gas conditions including at least one of: xenon gas flow and/or the remaining level of xenon gas.

16. The device of any one of paragraphs 13-15, wherein the medication event monitoring system is configured to activate with each device use.

17. The device of any one of paragraphs 13-16, wherein the medication event monitoring system further includes a wireless connection.

18. The device of paragraph 17, wherein the wireless connection includes at least one of a Bluetooth™ connection and a Wi-Fi connection.

19. A self-contained breathing apparatus for on-demand inhalation of a therapeutic xenon gas, the apparatus comprising: (i) a replaceable canister including a mixture of high-pressure gas that contains air and a therapeutic xenon gas, the xenon gas being in the range of about 0.5-35% of the mixture; (ii) a built-in regulator connected to the canister and configured to receive and transform the mixture from the high-pressure gas to a breathable level gas; (iii) a face mask connected to the built-in regulator, the face mask receiving the breathable level gas of the mixture; (iv) a push-button mounted to the canister and configured to activate, upon pressing, flow of the mixture from the canister to the face mask; (v) a graded flowmeter readout connected to the canister and indicative of changes in the mixture, the changes including flow of the mixture and a remaining amount of the mixture in the canister; and (vi) a medication event monitoring system (MEMS) mounted at least in part on the canister, the MEMS being communicatively coupled to the canister and to a remote computer, the MEMS being activated with each pressing of the push-button, the MEMS reporting one or more use aspects of the mixture.

20. A method for treating or preventing an act of suicide, homicide or self-harming behavior, the method comprising: administering a xenon composition to a subject, thereby treating or preventing the act of suicide, homicide, or self-harming behavior.

21. The method of paragraph 20, wherein the subject is determined to be at high risk for suicide, homicide or self-harming behavior.

22. The method of paragraph 21, wherein the subject is determined to be at high risk for suicide, homicide or self-harming behavior based on self-reporting by the subject or as diagnosed by a healthcare professional using a clinical test.

23. The method of any one of paragraphs 20-22, wherein the subject is diagnosed as having a high risk for suicide when the subject has a Columbia Suicide Severity Risk Scale (C-SSRS) level of at least Category 5.

24. The method of paragraph 22, wherein the subject is diagnosed as having a high risk for homicide when the subject is determined to have a Key to Danger level of at least 4 on the Centers for Disease Control and Prevention's Danger Assessment Test.

25. The method of paragraph 22, wherein the subject is diagnosed has having a high risk for self-harming behavior based on a past self-harming event or a score of at least 1 on the Self-Harm Inventory test.

26. The method of any one of paragraphs 20-25, wherein the subject is in emotional and/or physical distress.

27. The method of any one of paragraphs 20-26, wherein the subject is interrupted in an attempt of suicide, homicide or self-harming behavior.

28. The method of any one of paragraphs 20-27, wherein the subject is holding a means for suicide, homicide, or self-harming behavior.

29. The method of paragraph 28, wherein the means for suicide, homicide, or self-harming behavior comprises a firearm, a cutting utensil, a poison, a vial of drugs, an accelerant, an ignition source, a flammable liquid or gas, an asphyxiant, access to a ledge, bridge, pillar, or outcropping for jumping from a height, a ligature, a noose, material for electrocution, explosive material, access to a road or freeway for vehicular suicide, access to train tracks, or access to water for drowning.

30. The method of any one of paragraphs 20-29, wherein the subject is further administered an antidepressant, anti-anxiety, or anti-psychotic medication.

31. The method of any one of paragraphs 20-30, wherein the antidepressant, anti-anxiety, or anti-psychotic medication is formulated for aerosol delivery.

32. The method of any one of paragraphs 20-31, wherein the xenon composition is self-administered or administered by another individual.

33. The method of any one of paragraphs 20-30, wherein the xenon composition comprises 0.5 to 35% xenon.

34. A method of decreasing suicidal ideation in a subject having enhanced levels of at least one metabolite generated by indolamine 2, 3 deoxygenase comprising administration of 0.5-35% xenon gas.

35. A method of decreasing suicidal ideation in a subject comprising: (i) measuring levels of at least one metabolite generated by indolamine 2, 3, deoxygenase in a biological sample obtained from a subject; and (ii) administering 0.5-35% xenon gas to the subject, thereby decreasing suicidal ideation.

36. The method of paragraph 34 or 35, wherein the at least one metabolite is quinolinic acid or kynurenine.

37. The method of any one of paragraphs 34-36, wherein the enhanced levels are higher than age-matched controls.

38. The method of any one of paragraphs 34-37, wherein the xenon composition is administered by inhalation.

1. A method for reducing the intensity of thoughts and/or feelings associated with suicide, self-harm, or homicide, the method comprising: administering a xenon composition to a subject in need thereof, thereby reducing the intensity of the thoughts and/or feelings.

2. The method of claim 1, wherein the xenon composition is self-administered.

3. The method of claim 1, wherein the xenon composition is administered by a second individual.

4. The method of claim 3, wherein the second individual comprises an emergency responder, a friend, a relative, a co-worker, or a health care professional.

5. The method of claim 1, wherein the xenon composition comprises at least 0.5-35% xenon by volume.

6. (canceled)

7. The method of claim 1, wherein the administration of xenon is a single exposure to xenon.

8. The method of claim 1, wherein the administration of xenon is repeated as necessary until the intensity of the thoughts and/or feelings is reduced by at least 20%.

9. (canceled)

10. (canceled)

11. The method of claim 1, wherein the xenon composition is administered by inhalation.

12. (canceled)

13. A device for self-administration of xenon, the device comprising:
(a) a canister containing a xenon gas stored at a high-pressure level;
(b) a regulator in which the xenon gas is transformed from the high-pressure level to a breathable level;
(c) a face mask communicatively coupled to the canister and the regulator and through which the xenon composition is externally administered, the face mask being connected to the canister and the regulator such that the xenon gas flows from the canister, through the regulator, and into the face mask; and
(d) a medication event monitoring system communicatively coupled with the canister and configured to detect and/or determine changes in the xenon gas.

14. The device of claim 13, wherein the device further comprises a push button configured to activate xenon gas flow from the canister to the face mask.

15. The device of claim 13, further comprising a graded flowmeter readout indicative of xenon gas conditions including at least one of: xenon gas flow and/or the remaining level of xenon gas.

16. The device of claim 13, wherein the medication event monitoring system is configured to activate with each device use.

17. (canceled)

18. (canceled)

19. A self-contained breathing apparatus for on-demand inhalation of a therapeutic xenon gas, the apparatus comprising:
(i) a replaceable canister including a mixture of high-pressure gas that contains air and a therapeutic xenon gas, the xenon gas being in the range of about 0.5-35% of the mixture;
(ii) a built-in regulator connected to the canister and configured to receive and transform the mixture from the high-pressure gas to a breathable level gas;
(iii) a face mask connected to the built-in regulator, the face mask receiving the breathable level gas of the mixture;
(iv) a push-button mounted to the canister and configured to activate, upon pressing, flow of the mixture from the canister to the face mask;
(v) a graded flowmeter readout connected to the canister and indicative of changes in the mixture, the changes including flow of the mixture and a remaining amount of the mixture in the canister; and
(vi) a medication event monitoring system (MEMS) mounted at least in part on the canister, the MEMS being communicatively coupled to the canister and to a remote computer, the MEMS being activated with each pressing of the push-button, the MEMS reporting one or more use aspects of the mixture.

20. A method for treating or preventing an act of suicide, homicide or self-harming behavior, the method comprising: administering a xenon composition to a subject, thereby treating or preventing the act of suicide, homicide, or self-harming behavior.

21. The method of claim 20, wherein the subject is determined to be at high risk for suicide, homicide or self-harming behavior.

22.-26. (canceled)

27. The method of claim 20, wherein the subject is interrupted in an attempt of suicide, homicide or self-harming behavior.

28. The method of claim 20, wherein the subject is holding a means for suicide, homicide, or self-harming behavior.

29. (canceled)

30. The method of claim 20, wherein the subject is further administered an antidepressant, anti-anxiety, or anti-psychotic medication.

31. (canceled)

32. The method of claim 20, wherein the xenon composition is self-administered or administered by another individual.

33. The method of claim 20, wherein the xenon composition comprises 0.5 to 35% xenon.

34.-38. (canceled)
